(19) United States
(12) Patent Application Publication
(10) Pub. No.: US 2005/0228277 A1
(43) Pub. Date: Oct. 13, 2005

(54) System and method for 2D partial beamforming arrays with configurable sub-array elements

(75) Inventors: Stephen R. Barnes, Bellevue, WA (US); Mirsaid S. Bolorforosh, Portola Valley, CA (US); Robert N. Phelps, Fall City, WA (US)

Correspondence Address: Siemens Corporation;Intellectual Property Department, 170 Wood Avenue South, Iselin, NJ, 08830, US

(73) Assignee: Siemens Medical Solutions USA, Inc.

(21) Appl. No.: 10/818,923

(22) Filed: Apr. 5, 2004

(51) Int. Cl. A61B 8/00

(52) U.S. Cl. 600/437; 600/459

(57) ABSTRACT

Methods and systems for electronically scanning within a three dimensional volume while minimizing the number of system channels and associated cables connecting a two-dimensional array of elements to an ultrasound system are provided. Larger apertures can be utilized with existing 2D transducer electronics, whose purpose is to reduce the number of conductors in the transducer cable, by having the partially beam formed sub-arrays consist of a sub-array(s) of configurable elements. Exemplary 2D transducer electronics include electronics for the entire beam forming process, partial beam forming, e.g. delaying in time and summing of signals, walking aperture multiplexing, e.g. sequential sub-array actuation, sub-aperture mixing, e.g. delaying in phase and summing, time division multiplexing, e.g. sub-dividing and allocating available bandwidth as a function of time, and frequency division multiplexing, e.g. sub-dividing and allocating available bandwidth as a function of frequency.

### BACKGROUND

Typical aperture sizes for two-dimensional diagnostic ultrasound transducers range anywhere from 30 wavelengths by 30 wavelengths up to 30 wavelengths by 200 wavelengths. For example, a two-dimensional array has on the order of 60 by 60 to 60 by 200 spatial sampling locations or elements. Accordingly, such two-dimensional arrays have from 4,000 to 12,000 elements.

Typical high performance medical diagnostic ultrasound systems have about 200 beamforming channels and an associated 200 signal conductors in the transducer cable connecting the beamforming channels to the transducer array. Currently, 4,000+ transmission lines are not provided in a clinically useful cable. Therefore, current ultrasound systems and transducers may not be capable of real-time electronic, fully sampled three-dimensional beam formation without significantly sacrificing image quality or clinical usefulness.

Accordingly, there is a need for an ultrasound system and transducer capable of real-time electronic, fully sampled three-dimensional beam formation without significantly sacrificing image quality or clinical usefulness.

### SUMMARY

The present invention is defined by the following claims, and nothing in this section should be taken as a limitation on those claims. By way of introduction, the preferred embodiments described below relate to a multi-dimensional transducer array system for ultrasonically scanning a three dimensional volume. The system includes a multi-dimensional array of configurable sub-sets, each configurable sub-set comprising a plurality of transducer elements, each of the transducer elements capable of being selectively interconnected with at least another of the transducer elements to form at least one macro-element of a plurality macro-elements. The system also includes a plurality of system channels coupled with the transducer elements and a processor coupled with the multi-dimensional array and the plurality of system channels and operative to configure the interconnection of the plurality of transducer elements of at least two of the plurality of sub-sets to form the at least one macro-element for each of the at least two of the plurality of sub-sets as a function of a beam position, the at least one macro-element of a first of the at least two of the plurality of sub-sets operative to generate a first signal and the at least one macro-element of a second of the at least two of the plurality of sub-sets operative to generate a second signal, and wherein the processor is further operative to combine the first and second signals for communication over one of the plurality of system channels.

The preferred embodiments further relate to a method for ultrasonically scanning a three dimensional volume in a multi-dimensional transducer array system.

In one embodiment, the method comprises: providing a multi-dimensional array of configurable sub-sets, each configurable sub-set comprising a plurality of transducer elements, each of the transducer elements capable of being selectively interconnected with at least another of the transducer elements to form at least one macro-element of a plurality macro-elements; providing a plurality of system channels coupled with the transducer elements; configuring the interconnection of the plurality of transducer elements of at least two of the plurality of sub-sets to form the at least one macro-element for each of the at least two of the plurality of sub-sets as a function of a beam position, the at least one macro-element of a first of the at least two of the plurality of sub-sets generating a first signal and the at least one macro-element of a second of the at least two of the plurality of sub-sets generating a second signal; and combining the first and second signals and communicating the combined first and second signals over one of the plurality of system channels.

Further aspects and advantages of the invention are discussed below in conjunction with the preferred embodiments.

### BRIEF DESCRIPTION OF THE DRAWINGS

FIG. 1 depicts a block diagram of an exemplary multi-dimensional transducer array system according to one embodiment.

FIG. 2 depicts a block diagram of an exemplary configurable 2D array for use with the system of FIG. 1, according to one embodiment.

FIGS. 3A-3F depict block diagrams of exemplary macro-element configurations for use with the system of FIG. 1, according to one embodiment.

FIG. 4 shows a block diagram of a 2D transducer array according to an alternate embodiment, for use with the system of FIG. 1.

FIGS. 5A-5C show an exemplary 2D array using time division multiplexing according to one embodiment, for use with the system of FIG. 1.

FIGS. 6A-C show an exemplary 2D array using time division multiplexing according to another embodiment, for use with the system of FIG. 1.

### DETAILED DESCRIPTION OF THE DRAWINGS AND PRESENTLY PREFERRED EMBODIMENTS

Methods and systems for electronically scanning within a three dimensional volume while minimizing the number of system channels and associated cables connecting a two-dimensional array of elements to an ultrasound system are provided. Larger apertures can be utilized with existing 2D transducer electronics, whose purpose is to reduce the number of conductors in the transducer cable, by having the 2D transducer electronics sub-channels connect to configurable macro-elements rather than non-configurable fixed elements. Exemplary 2D transducer electronics include electronics for the entire beam forming process, partial beam forming, e.g. delaying in time and summing of signals, walking aperture multiplexing, e.g. sequential sub-array actuation, sub-aperture mixing, e.g. delaying in phase and summing, time division multiplexing, e.g. sub-dividing and allocating available bandwidth as a function of time, and frequency division multiplexing, e.g. sub-dividing and allocating available bandwidth as a function of frequency.

One approach to three-dimensional imaging uses beamforming electronics within the transducer to avoid a large number of transmission lines in the cable or a large number of beamforming channels in the system. For example, the entire beamforming process may occur in the transducer. However, beam forming circuitry has a high degree of complexity in terms of both the number of circuit functions, number of components and cost, and locating such circuitry, especially in its entirety, in the transducer further stresses the transducer's physical constraints, such as size, e.g. ergonomic and manufacturability, power and thermal limitations.

Other approaches attempt to reduce the number of necessary cables without substantially sacrificing functionality by dividing the beam forming circuitry between the transducer and the system unit, the division being made so as to minimize the number of necessary independent interconnection between the transducer and the ultrasound system unit. Such approaches, however, still require changes in or augmentation of the beam forming process to account for the lower bandwidth between the transducer and the system unit. For example, one approach uses a sparse array for three-dimensional imaging to reduce the number of transmission lines used in a cable. The system disclosed in U.S. Pat. No. 6,279,399, entitled “MULTI-DIMENSIONAL TRANSDUCER ARRAY APPARATUS”, issued on Apr. 28, 2001, herein incorporated by reference, uses a combination of a sparse array for three-dimensional imaging and a configuration of elements for two-dimensional imaging. A set of mode switches or multiplexers configure the transducer elements to form either a one-dimensional array providing a two-dimensional scan mode or a two-dimensional sparse array providing a three-dimensional scan mode. Sparse array configurations utilize a limited set of transducer elements from the full two-dimensional arrangement of elements of the two-dimensional array. A typical sparse array configuration could contain between 256 and 512 transducer elements which would be utilized for three-dimensional (3D) scanning. The arrangement of the transducer elements in a sparse array can be in various formats, such as, randomly selected, randomly selected within the constraints of a binned pattern, periodic patterns with different periodicity for the transmitter and receiver elements, algorithmically optimized patterns from computer optimization, or a combination of periodic and algorithmically optimized patterns. In the two-dimensional scan mode, the length of the sparse elements is extended in one direction, forming a conventional one-dimensional array for two-dimensional images in a single fixed image plane. However, sparse arrays for three dimensional imaging have poor sensitivity and contrast resolution.

Other approaches to controlling a large number of elements using a minimal number of cable conductors include partial beam forming, walking aperture multiplexing, sub-aperture mixing, time division multiplexing and frequency division multiplexing.

Partial beamforming is described in U.S. Pat. No. 6,126,602, entitled “PHASED ARRAY ACOUSTIC SYSTEMS WITH INTRA-GROUP PROCESSORS”, issued on Oct. 3, 2000, herein incorporated by reference. In partial beamforming, circuitry in the transducer identifies elements having substantially similar beam forming time delays. These elements are driven by a common signal and signals received from these elements are delayed to align them in time and then summed at the transducer to be sent over a single conductor. However, the combining/summing of signals at the transducer prevents those signals from being distinguished by the system and therefore constrains the sensitivity and resolution, especially away from the partial beamforming focus position. Typically, this cannot be fixed because the system is unable to distinguish the original signals once they are combined.

Walking aperture multiplexing is described in U.S. Pat. No. 6,238,346, entitled “SYSTEM AND METHOD EMPLOYING TWO DIMENSIONAL ULTRASOUND ARRAY FOR WIDE FIELD OF VIEW IMAGING”, issued on May 29, 2001, herein incorporated by reference. In walking aperture multiplexing, the array of elements is divided into a series of sub-arrays, arranged in an ordered sequence. During operation of the transducer, each sub-array is actuated in turn sequentially. However, the number of system channels and wires connecting the transducer to the system limits the aperture size. Based on the typical ratio of elements in 2D array to the number of system channels, the apertures would be too small to provide adequate resolution.

Sub-aperture mixing is described in U.S. Pat. No. 5,573,001, entitled “ULTRASONIC RECEIVE BEAMFORMER WITH PHASED SUB-ARRAYS”, issue on Nov. 12, 1996, the disclosure of which is incorporated herein by reference. Sub-aperture mixing uses partial beamforming, generally described above, to combine signals from multiple elements for processing by a single receive beamformer channel. Signals from different elements are mixed with signals having selected phases, and the mixed signals are then summed together to form a partially beam formed sub-array signal. The sub-array signal is responsive to each of the plurality of elements and may be processed with a single receive beamformer channel. Sub-array mixing across an array allows the use of more elements than receive beamformer channels. However, as with partial beamforming, the combining/summing of signals at the transducer prevents those signals from being distinguished by the system and therefore constrains the sensitivity and resolution.

Time division multiplexing (“TDM”) and frequency division multiplexing (“FDM”) are both methods of better utilizing the available bandwidth of the available cable conductors by sharing that bandwidth among the various transmitters and receivers that need it. TDM is a method of putting multiple data streams in a single signal by separating the signal into many segments, each having a very short duration. Each individual data stream is reassembled at the receiving end based on the timing. A multiplexer accepts the input from each individual end user, breaks each signal into segments, and assigns the segments to the composite signal in a rotating, repeating sequence. The composite signal thus contains data from multiple senders. At the other end of the long-distance cable, the individual signals are separated out by means of a de-multiplexer, and routed to the proper end users. A two-way communications circuit requires a multiplexer/de-multiplexer at each end of the cable. In ultrasound, TDM may be used to send more element control signals, either digital or analog, over a limited number of channels, however, as the element control signals are broken up across time slots in the TDM scheme, high frequency simultaneous control of multiple elements is limited by the throughput of the cable and the associated multiplexers and de-multiplexers. See for example, U.S. Pat. No. 5,622,177, entitled “ULTRASOUND IMAGING SYSTEM HAVING A REDUCED NUMBER OF LINES BETWEEN THE BASE UNIT AND THE PROBE”, issued on Apr. 22, 1997, herein incorporated by reference.

FDM is a scheme in which numerous signals are combined for transmission on a single communications line or channel. Each signal is assigned a different frequency (sub-channel) within the main channel. As with TDM, a multiplexer circuit is required to combine the transmitted signals and a de-multiplexer is required to separate the received signals. In ultrasound, FDM may be used to send more element control signals over a limited number of channels, however, the bandwidth of each cable conductor still limits the number of simultaneous control signals that can be carried.

Sub-array mixing or partial beamforming may be desired in some situations and undesired in others. Multiplexing may be desired in some situations, but undesired in others. For example, multiplexing may not reduce the number of receive beamformer channels needed as compared to the number of elements.

Further, for all of the described approaches using 2D array electronics, the size of the active aperture supported by any particular physical implementation of 2D array transducer electronics is limited by the number of acoustic elements addressable by the transducer electronics and the physical size of the acoustic elements.

There are image quality tradeoffs involved in determining the physical size of the acoustic elements. Larger physical elements allow larger total array apertures to be formed which improves the spatial resolution of the image. However this also decreases the angular width of the diffraction pattern of the element and angular separation between the main lobe and grating lobes of the ultrasound beam. The result is a reduction in the maximum scan angle or higher grating lobe artifacts. When larger element sizes are used the grating lobes will be lower if smaller scan angles are used.

Another limitation of the prior art of 2D array transducer electronics is that the electronics dissipate a considerable amount of power, so that the thermal conditions within the transducer can limit the number of elements supportable by the electronics.

As opposed to adding electronics to the transducer to control the array elements, configurable arrays provide switching networks which configurably interconnect combinations of elements into one or more “macro-elements” connected with a given channel at any given time. In contrast to including 2D array electronics in the transducer, the thermal dissipation of the switches used to configurably interconnect elements can be quite low.

In U.S. Pat. No. 5,563,346, entitled “METHOD AND DEVICE FOR IMAGING AN OBJECT USING A TWO-DIMENSIONAL ULTRASONIC ARRAY”, issued on Oct. 8, 1996, herein incorporated by reference, three-dimensional scanning is provided using a minimum number of signal lines. A two-dimensional array operates as a linear, annular array to form beams normal to the array surface at different locations on the two-dimensional array. Concentric rings of elements are interconnected using a multiplexer or switching. Each concentric ring represents common delay areas for beamforming, so connects with a single signal line. However, the normal beam constraint limits the volume which can be scanned by the aperture size and shape of the two-dimensional array. Further, the disclosed configurable annular array can only form a single transmit-receive beam and cannot support the simultaneous formation of multiple receive beams, thus the frame rate is slower than the electronic 2D array methods described below by a factor equal to the number of simultaneous receive beams supportable by the electronic implementation.

U.S. Pat. No. 6,676,602, entitled “TWO DIMENSIONAL ARRAY SWITCHING FOR BEAMFORMING IN A VOLUME”, issued on Jan. 13, 2004, herein incorporated by reference, also discloses configurable arrays. In particular, an array of semiconductor or micro-machined switches electronically interconnect various elements of the two-dimensional array. Elements associated with a substantially same time delay are connected together as a macro-element, reducing the number of independent elements to be connected to beamforming or system channels. To beam form in the desired direction, the macro-elements are configured as a phased array or along substantially straight lines in at least two dimensions (i.e. along the face of the two-dimensional transducer). Such macro-elements allow transmission and reception along beams that are at an angle other than normal to the two-dimensional transducer array. Beams at such angles may be used to acquire information beyond the azimuth and elevation extent of the two-dimensional array. Various configurations of macro-elements are possible. For example, the macro-elements in each configuration are parallel across the two-dimensional array, but different configurations are associated with rotation of the macro-elements such that each configuration is at a different angle on the two-dimensional array. As another example, the macro-elements are configured in a plurality of separate rows of parallel macro-elements (i.e. configured as a 1.25D, 1.5D or 1.75D array of macro-elements). Two or more switches are provided for each system channel, allowing for rotation of macro elements. The different rotation positions of macro-elements defines different two-dimensional scan planes within the three-dimensional volume. Two, three or more switches are provided for each element to interconnect the elements in many possible combinations.

For high frame-rate 3D ultrasonic imaging multiple receive beams are simultaneously formed for each transmit beam. This allows the volume to be sampled more rapidly. A somewhat wider transmit beam is formed and narrower receive beams are simultaneously formed sampling the space insonified by the transmit beam. However, the disclosed configurable phased array can only support simultaneous receive beams in one plane set by the configured 1D phased array orientation. In rough comparison to the electronic 2D array methods described above, if the electronic 2D array method can support n simultaneous receive beams, the configurable phased array could support n simultaneous receive beams yielding a frame rate n slower. In addition, in general the electronic 2D array methods described above will provide 2D dynamic focusing, whereas the configurable phased array will provide 1D dynamic focusing in one direction and fixed focusing in the orthogonal direction.

In one embodiment, a 2D array which includes configurable elements, i.e. elements which may be configurably interconnected with each other and with a given channel, is provided. The interconnection of two or more configurable elements form a “macro-element,” also referred to as “virtual element.” In an alternate embodiment, possible macro-elements may include only single element of the configurable elements. The configurable 2D array is further coupled with 2D array electronics, as described above, thereby achieving the benefit of a larger aperture without substantially reducing frame rates or otherwise impeding the functionality of the transducer. Herein, the phrase “coupled with” is defined to mean directly connected to or indirectly connected through one or more intermediate components. Such intermediate components may include both hardware and software based components. By allowing the configurable formation of macro-elements, e.g. the interconnection of two or more elements, the necessary bandwidth between the transducer and the system unit may be reduced so as to be supportable by the combination of the 2D electronics implementations noted above and a clinically useful/practical cable/connection arrangement. It will be appreciated that, while the disclosed embodiments refer to a cable and associated electrical signal conductors which interconnect a transducer with an ultrasound system unit, the disclosed embodiments are applicable to any medium of interconnection characterized by less bandwidth than which is necessary to support simultaneous addressability of all of the available acoustic elements of the transducer, including optical interconnections, wireless interconnections using RF or infrared, or other interconnection technologies presently available or later developed, and all such applications are contemplated.

A macro-element is formed by connecting more than one small adjacent, either abutting or diagonally, configurable 2D array elements together. In an alternate embodiment, the interconnected elements may be only substantially adjacent or within close proximity. Further, in another alternate embodiment, elements may be selected for interconnection without regard to their proximity. In yet another alternative embodiment, macro-elements may consist of only a single element. It will be appreciated that increasing the number of available distinct interconnection configurations may increase the number of switches that are required, and or the complexity of the switching network, adding to the overall complexity and cost of the transducer. In one embodiment which isolates and reduces the number of required switches and reduces the complexity of the switching network, the configurable elements of the transducer array are sub-divided into sub-sets, the configurable transducer elements of each sub-set, also referred to as a configurable sub-set, being interconnectable with each other but not with the transducer elements of another sub-set. In an alternate embodiment, the sub-sets may overlap, partially or entirely, allowing interconnections among the configurable elements of those sub-sets that overlap each other. In another alternate embodiment, the particular elements included within any given sub-set may be dynamically modified. The 2D array may further be divided into sub-arrays, each sub-array including one or more sub-sets of interconnectable/configurable transducer elements thereby allowing for sub-arrays of macro-elements. In one embodiment, two or more sub-arrays may overlap, i.e. share one or more sub-sets, simultaneously or dynamically over time. In an alternate embodiment, the particular sub-sets included within any given sub-array may be dynamically modified.

In one exemplary embodiment, by arranging the connection so that the macro-element is narrow in the transmit beam steering direction and longer in the direction orthogonal to the beam steering, the diffraction pattern of the macro-element will be wide in the direction of the transmit beam to support this beam steering and narrower in the direction orthogonal to the direction of the transmit beam steering but still wider than the transmit beam supporting the receive beam steering in two directions. Once the transmit-receive event is completed, the configurable elements may be reconfigured into a different macro-element for a new transmit beam direction. A 2D array could thus become a reconfigurable 2D array of small macro-elements with further processing by 2D array electronics within the transducer. This allows the 2D array electronics to support a larger physical aperture, or, alternatively, to support a more finely divided aperture, without substantial increases in thermal dissipation.

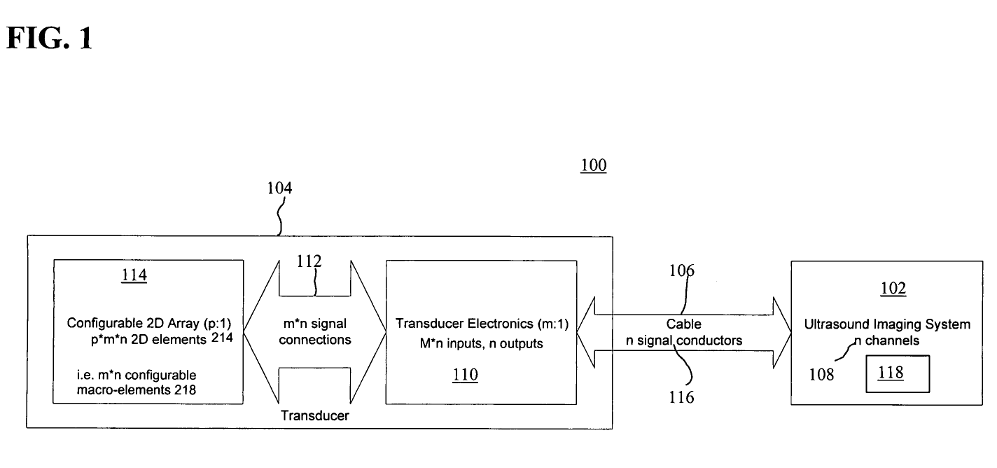

FIG. 1 shows a block diagram of an exemplary multi-dimensional transducer array system 100 for ultrasonically scanning a three dimensional volume. The system 100 includes ultrasound system unit 102 having n system channels 108 and a beamformer 118, a configurable 2D transducer 104, and an interconnecting cable 106 having n signal conductors 116. The interconnecting cable 106 may further include additional conductors for carrying control signals and other purposes (not shown). In one embodiment, there is a one to one relationship between the number of signal conductors 116 and the number of system channels 108. In systems using TDM or FDM schemes, there may be more system channels 108 than signal conductors 116. The system channels 108 represent the number of independent data signals capable of being generated and communicated over the physical connection between the configurable transducer 104, via the transducer electronics 110, and the system unit 102, e.g. the conductors 116 of the cable 106. For purposes of the disclosed embodiments, a reference to the system channels 108 includes the associated physical medium, such as the associated conductor(s) 116 of the cable 106.

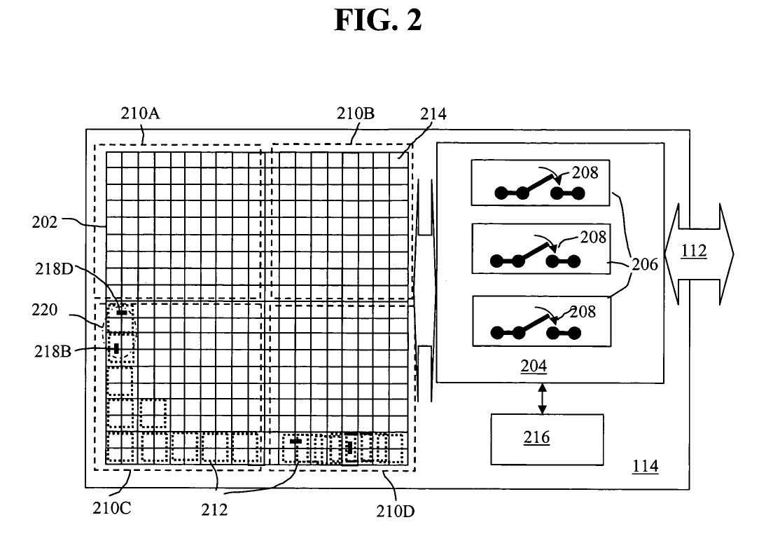

The configurable 2D transducer 104 includes transducer electronics 110, a configurable 2D transducer array 114 (shown in more detail in FIG. 2), and sub-channels 112 which interconnect the electronics 110 and configurable array 114. The number of sub-channels 112 exceeds the number of signal conductors 116 in the interconnecting cable 106 and may exceed the number of system channels 108.

Referring to FIG. 2, the configurable array 114 includes an array 202 of transducer elements 214, the number of which exceeds the number of system channels 108, signal conductors 116 in the interconnecting cable 106 and sub-channels 112 between the transducer electronics 110 and configurable array 114.

FIG. 2 further shows a block diagram of an exemplary configurable 2D array 114 for use with the disclosed embodiments. The configurable 2D array 114 consists of the 2D array 202 of acoustic elements 214, an array/network 204 of switches 208, grouped in sets 206, with more than one switch 208 for every 2D array acoustic element 214, and a switch controller 216 coupled with the switch network 204. The switch network 204 interconnects the sub-channels 112 with the 2D array 202 as will be described. An exemplary 2D array for use with the disclosed embodiments is detailed in U.S. Pat. No. 6,676,602, referenced above. The switches can be fabricated using micromachining techniques (MEMS, micro-electro-mechanical systems), or they could be analog semiconductor switches. See for example, U.S. patent application Publication No. 2003/0032211 A1, entitled “MICROFABRICATED TRANSDUCERS FORMED OVER OTHER CIRCUIT COMPONENTS ON AN INTEGRATED CIRCUIT CHIP AND METHODS FOR MAKING THE SAME”, published Feb. 13, 2003, herein incorporated by reference.

In an alternate embodiment, a portion of the transducer electronics 110 is coupled between the 2D acoustic array 202 elements 214 and the switches 208. For example a preamplifier and high voltage protection circuitry could be placed between the 2D acoustic array 202 elements and the switches 208 to mitigate the electrical loading of a high impedance 2D acoustic array element 214 by the interconnection and switches 208.

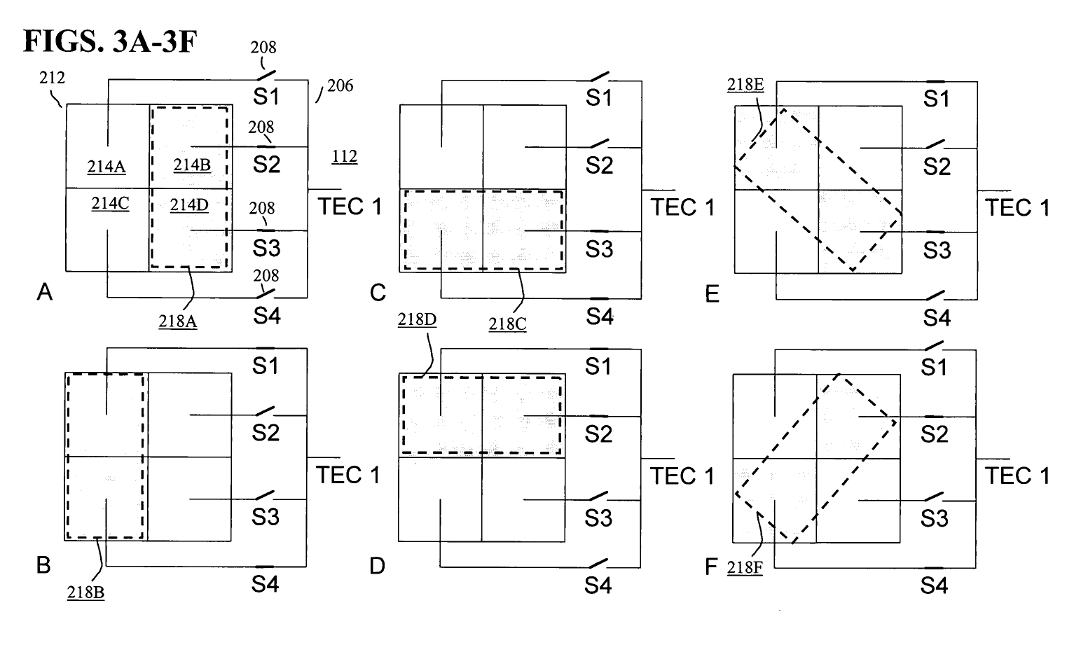

In one embodiment, a multi-dimensional transducer array system 100 for ultrasonically scanning a three dimensional volume is provided. The system includes a multi-dimensional, e.g. 1.25, 1.5, 1.75 or 2 dimensional, array 202, the elements of which are partitioned into sub-sets 212. Each sub-set 212 includes a plurality of transducer elements 214, each of the transducer elements 214 capable of being selectively, e.g. switchably, interconnected with each other to form one or more macro-elements 218A-218F. Accordingly, a sub-set 212 may be referred to as a “configurable” sub-set 212 and the elements 214 of the sub-set 212 may be referred to as “configurable” elements 214. The number of different macro-elements 218A-218F, i.e. the number of different sizes, shapes or orientations of the interconnected elements 214, that can be created is a function of the number of transducer elements 214 in the sub-set 212 and the number of elements 214 interconnected in each macro-element 218A-218F (exemplary possible macro-elements of a 2 by 2 sub-set 212 wherein each macro-element consists of two elements 214 are shown in FIG. 3. It will be appreciated that single element macro-elements are also possible). In one embodiment, each sub-set 212 can form one macro-element 218A-218F at any given time. In alternative embodiments, each sub-set 212 can form more than macro-element 218A-218F at any given time. Further, in one embodiment, each of the sub-sets 212 may form the same macro-element 218A-218F, i.e. form the same size, shape or orientation of interconnected elements 214, or, alternatively, different sub-sets 212 may form different macro-elements 218A-218F. For example some sub-sets 212 may form macro-elements 21A, 218B, 218E having a first orientation while other sub-sets 212 form macro-elements 218C, 218D and 218F having a second orientation 90 degrees rotated from the first orientation In another embodiment, the array 202 is further divided into a plurality of sub-arrays 210A-210D, each sub-array 210A-210D including one or more sub-sets 212, thereby allowing the formation of sub-arrays 210A-210D of macro-elements 218A-218F, each sub-array 210A-210D capable of forming a different groups 220 of macro-elements 218A-218F.

In one embodiment, a switching network 204 is coupled with the multi-dimensional array 202 and selectively interconnects the transducer elements 214 into a plurality of macro-element 218A-218F groups 220, a macro-element 218A-218F group 220 referring to a particular arrangement/formation of macro-element(s) 218A-218F formed by one or more sub-sets 212 at any given time. The switching network includes a plurality of switch sets 206, each which is associated with a sub-set 212 of transducer elements of the array 202 of transducer elements 214. Each switch set 206 includes a plurality of switches 208 which are capable of selectively interconnecting at least one transducer element(s) 214 of the associated sub-set 212 thereby creating a macro-element 218A-218F. Each macro-element group 220 includes a different configuration of switches 208 and accompanying interconnections of transducer elements 214.

As described above, the system further includes a plurality of system channels 108 coupled with the transducer electronics 110, for example via the signal conductors 116 of an interconnecting cable 106. The transducer electronics 110 are coupled with the configurable array 114 via sub-channels 112. As described herein, the transducer electronics 110 effectively bridge between the sub-channels 112 and the lesser number of system channels 108. The sub-channels 112 are coupled with the transducer elements 214 via the switch sets 206 of the switching network 204 wherein each sub-channel 112 connects with at least two of the switches 208 of a given switch set 206, i.e. each sub-channel 112 is coupled with at least one macro-element 218A-218F. Via this arrangement, each system channel 108 is capable of being coupled with a plurality of macro-elements 218A-218F, each macro-element 218A-218F including at least one element(s) 214, via the signal conductors 106, transducer electronics 110, sub-channels 112 and switching network 204.

The system 100 also includes a processor 110, 118 (including, in one embodiment, beamformer 118 and transducer electronics 110 as described in more detail below) coupled with the multi-dimensional array 202 and the plurality of system channels 108. The processor 110, 118 configures the interconnection of the plurality of transducer elements 214 of at least two of the plurality of sub-sets 212 to form macro-element(s) 218A-218F for each of the sub-set 212 as a function of a beam position, e.g. steering angle. For example, the processor 110, 118 causes the switching network 204 to form a first macro-element group 220 of the plurality of macro-element group 220 and generate a first signal to cause at least one macro-element 218A-218F of the first macro-element group 220 to either form a first transmit beam (if this is a transmit operation) or receive a first echo (if this is a receive operation). The signals are generated using one of the methods of beam forming described above, and in further detail below. In an exemplary scanning operation wherein the array 202 is further divided into sub-arrays 210A-210D, a first macro-element(s) 218A-218F formed by a sub-set 212 of one of the sub-arrays 210A-210D generates a first signal and another macro-element(s) 218A-218F of another sub-set 212 of the same or another sub-array 210A-210D generates a second signal. The processor 110, 118 further combines the first and second signals for communication to the system channels 108 over one of the plurality of cable conductors 116, as will be described in more detail below. In this way, a smaller number of system channels 108 may be used to communicate with a larger number of elements 214, as described.

The beamformer 118 generates the control signals (transmit signals) that cause the transducer array 202 to emit acoustic energy and receives and processes the signals (receive signals) generated by the array 202 in response to received acoustic echoes. In one embodiment, such as an embodiment which utilizes partial beamforming or sub-aperture mixing, the control signals control transmitters (not shown), also referred to as an intra-group transmit processor, located in the transducer electronics 110 which generate the actual excitation signals to the array 202 in response to the control signals from the beamformer 118. The receive signals are communicated between the system unit 102 and the transducer 104 via the system channels 108, interconnecting cable 106 and conductors 116. The beamformer 118 generates transmit control signals and processes receive signals via the system channels 108 and interconnecting cable 106 and signal conductors 116 in conjunction with the transducer electronics 110 as described herein. In an alternate embodiment, the beamformer 118 is encompassed by the transducer electronics 110 and wholly located in the transducer 104. As used herein the term “processor” refers to the combination of the beamformer 118 and transducer electronics 110 no matter how the functionality of the beamformer 118 and transducer electronics are partitioned/physically implemented between the transducer 104 and system unit 102. In conjunction with the 2D configurable array 114, the electronics 110, which may implement at least one of the beam forming or multiplexing methodologies described above and in more detail below, or other signal processing technique, in conjunction with the beamformer 118 to permit the system unit 102 and beamformer 118 to address substantially all of the transducer elements 214 using the available system channels 108 and signal conductors 116 without substantial loss of system 100 functionality, the system unit 102 and beamformer 118 being appropriately designed to utilize the transducer electronics 110. For example, some technologies used to implement configurable 2D arrays 114 may require substantially more voltage to operate as compared to conventional transducer technology, therefore the transducer electronics 110 would be appropriately implemented to handle the increased voltage requirements. Further, in implementing a given beam forming or multiplexing methodology, the electronics 110 and beam former 118 account for the characteristics of the configurable 2D array 114 when forming beams or processing received signals, so as take advantage of the enhanced functionality of the configurable 2D array 114 as well as compensate for the characteristics thereof. For example, the beam former 118 and electronics 110 must consider that the apparent acoustic source will move as the group 220 of macro-elements 218A-218F changes and that the directivity pattern of the macro-elements 218A-218F will change as the group 220 changes. Further, the beam former 118 and electronics 110 must determine which elements 214 to interconnect to form macro-elements 218A-218F to achieve a desired beam forming effect. The beam former 118 and electronics 110 are suitably designed/programmed to make such computations when beam forming.

An exemplary 2D transducer array 202 for use with the disclosed embodiments is a 64×64 element rectangular grid array 202 with a pitch of 300 μm (19.2 mm×19.2 mm), with 4,096-2D acoustic elements 214. This 2D grid pitch is λ/2 at 2.5 MHz. A 2:1 configurable array may use 8,192-switches and give 2,048-configurable elements, i.e. macro-elements 218A-218F. This could be supported by transducer electronics 110 consisting of 128-partial beamforming circuits (not shown inside the transducer electronics 110 block) each supporting beamforming 16-sub-channels 112.

FIGS. 3A-3F show a block diagram of an exemplary 2D transducer array 202 sub-set 212 having four 2D elements 214A-214D arranged 2 by 2 and accompanying switch set 206 of the switching network 204 showing various interconnection arrangements and resultant macro-elements 218A-218F, each having a size of two 2D elements 214A-214D. For example, in FIG. 3A, Switches 208 S2 and S3 are closed and switches S1 and S4 are open, thereby forming a macro-element from among elements 214B and 214D connected with the sub-channel 112 (referred to also as a transducer electronics channel (“TEC”)). FIGS. 3B-3F show the remaining possible combinations of 2 of 4 elements 214. For grouping 2D acoustic elements 214 into macro-elements 218A-218F there may be four switches 208 for each sub-set 212. There would be six selectable configurations of configurable elements where two adjacent 2D elements 214 are connected to one sub-channel 112, connecting the two adjacent 2D element 214 neighbors to support beamforming in the generally 0°, 45°, 90°, or 135° directions. It will be appreciated that the size of the sub-set 212 may be larger allowing for more possible sizes, shapes and orientations of macro-elements 218A-218F.

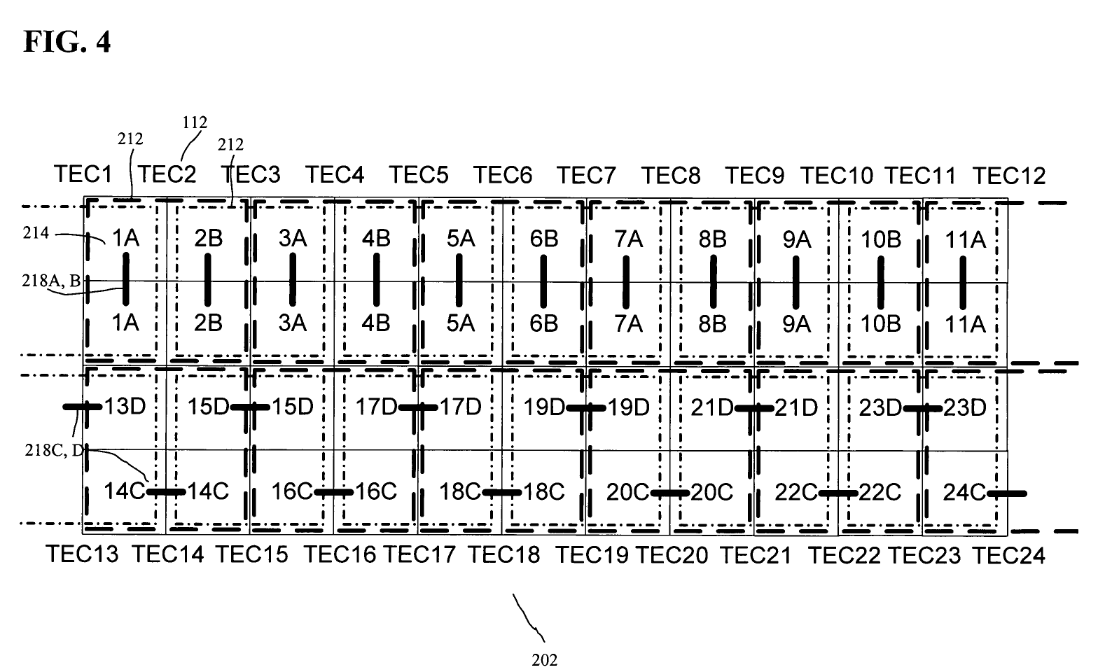

FIG. 4 shows an alternative embodiment of a 2D transducer array 202 having overlapping sub-sets 212, each sub-set 212 having four elements 214 configurable as shown in FIGS. 3A-3F. The regions of the 2D array 202 that adjacent sub-channels 112 would support overlap by two 2D array elements 214. Since these regions overlap by two 2D array elements 214, each 2D array element 214 has two single-pole-single-throw (“SPST”) switches 208 which select it to be connected to one of two possible sub-channels 112. Alternately a single single-pole-double-throw (“SPDT”) switch may serve the same function.

FIG. 4 further shows an arrangement of transducer electronics/sub-channels 112 and four macro-element 218A-218D configurations. In FIG. 4 the regions, i.e. sub-set 212 of 2D elements 214, covered by a particular sub-channel 112, labeled as “TECn”, are shown alternately by dashed lines or by dash-dot lines. In each of the 2D acoustic array elements 214 shown, the number-letter combinations are associated with sub-channel 112/configuration 220 combinations indicating which sub-channel 112 and macro-element 218A-218F group 220 (as shown in FIG. 3) is used for that element 214. The vertical bars in the upper half of the diagram indicate which 2D acoustic array elements 214 are connected together to form the macro-element 218A or 218B. The upper row of sub-channels 112 are shown with the macro-element 218A-218F group 220 for beamforming toward the right or left. The horizontal bars in the lower half of the diagram indicate which 2D acoustic array elements 214 are connected together. The lower row of sub-channels 112 are shown with macro-element 218A-218F group 220 for beamforming toward the top or bottom. For beamforming to the upper-right or lower-left, the macro-element labeled as 218E (shown in FIG. 3) would be used everywhere. For beamforming to the lower-right or upper-left, the macro-element labeled as 218F (shown in FIG. 3) would be used everywhere.

In one embodiment, the switches 208 are implemented as micro-mechanical (“MEM”'s) based devices and fabricated using integrated circuit manufacturing techniques. Integration of the 2D acoustic array 114, switches 208 and/or some or all of the transducer electronics 110 may be accomplished on a single MEMS substrate. For example, switches 208 may be implemented as capacitive membrane switches that may be co-fabricated with a capacitive membrane ultrasound transducer, CMUT. U.S. patent application Publication No. 2003/0032211 A1, referenced above, teaches how to fabricate silicon dioxide membrane CMUT's over the top of an electronic circuit on a silicon wafer. Similar techniques could allow the co-fabrication of the switches 208 and CMUT's over the electronic circuits. Alternatively semiconductor switches could be included in the electronic circuits, and CMUT's could be fabricated on top.

Other aspects of the system 100 as disclosed include allowing the processor 110/118 to configure the configurable array 114 for a given transmit operation differently than the corresponding receive operation. For example, the processor may be operative to cause the switching network 204 to form a first macro-element 218A-218F group 220 when generating a first signal to cause the at least one macro-element 218A-218F to form the first beam and to cause the switching network 204 to form a second macro-element 218A-218F group 220, different from the first macro-element 218A-218F group 220, when generating a second signal to cause at least one macro-element 218A-218F of the second macro-element 218A-218F group 220 to receive the first echo.

In another embodiment, the system 100 is capable of configuring a sparse array pattern, as detailed above, using a group 220 of macro-elements 218A-218F.

As was described above, the disclosed embodiments combine cable conductor 116 reducing electronics 110/beam former 118 methodologies with a configurable array 114. For example, a configurable array 114 may be combined with one of walking aperture multiplexing, partial beam forming, sub-aperture mixing, time division multiplexing, or frequency division multiplexing, or combinations thereof.

In one embodiment implementing walking aperture multiplexing, the array 202 is sub-divided into two or more sub-arrays 210A-210D, where the processor 118/110 sequentially actuates (receive or transmit) each of the sub-arrays 210A-210D sequentially, each element 214 of the sub-array 210A-210D being configured into a particular macro-element 218A-218F group 220.

For example, the multi-dimensional array 202 may include N×M transducer elements 214, there being M columns of N transducer elements 214, wherein M and N are integers. The processor 118/110 includes a transmitter (not shown) for generating a first signal to cause at least one macro-element 218A-218F to form a first beam and a receiver (not shown) for generating the first signal to cause the macro-element 218A-218F to receive a first echo. The processor 118/110 is further operative to couple the transmitter with a plurality of sub-arrays 210A-210D of N×X transducer elements 214, where X is an integer less than M, each of the plurality of sub-arrays 210A-210D comprising at least one sub-set 212 of elements 214, so as to cause each of the at least one sub-set 212 of each sub-array 210A-210D to form a macro-element 218A-218F groups 220 and cause at least one macro-element 218A-218F to form a beam. The processor 118/110 sequentially couples the transmitter and receiver with each of the sub-arrays 210A-210D so as to enable reception by the receiver of echoes from an elongated sector volume.

In embodiments using signal mixing techniques, such as partial beam forming or sub-aperture mixing, the processor 118/110 combines signals transmitted to/received from a first set of macro-elements 218A-218F of a given macro-element 218A-218F group 220 with signals transmitted to/received from a second set of macro-elements 218A-218F and conveys the combined signals over one of the plurality of system channels 108.

In partial beam forming, the processor 110/118 combines one signal with another signal by delaying the first signal with respect to the second signal and summing the delayed first signal with the second signal. As described above, a portion of this beamforming process may occur in the transducer electronics 110 and the remainder of the process may occur in the system beamformer 118. For example, the array 202 of macro-elements 218A-F is further divided into a plurality of sub-arrays 210A-210D of macro-elements 218A-F, each of the plurality of sub-arrays 210A-210D comprising at least one sub-set 212 of transducer elements 214. As described above, sub-arrays 210A-210D may overlap. The processor 110/118 also includes a plurality of intra-group transmit processors (not shown) coupled with the plurality of sub-arrays 210A-210D which operate to cause the formation of a beam directed into a region of interest. The array 202 of transducer elements 214 further includes transducer elements 214, including at least one configurable sub-set 212 of elements 214, configured to receive echoes. The transducer electronics 110 includes a receive beamformer (not shown) which includes the sub-channels 112, each of the sub-channels 112 including a beamformer delay (not shown) operative to synthesize receive beams for each sub-array 210 from the received echoes by delaying the signal received from the macro-element 218A-218F of the configurable sub-set 212 of elements 214 configured to receive, where each receive beamformed signal from each sub-array 210 is sent to the system 102 via a cable conductor 116 (system channel 108). The receive beamformer 118 further includes a beamformer summer (not shown) which receives and sums the signal from the system channels 108 and an image generator (not shown) operative to form an image of the region of interest based on the signals received from the receive beamformer.

In sub-aperture mixing, the processor 110/118 combines one signal with another signal by altering the phase of the first signal with respect to the second signal and summing the altered first signal with the second signal. A portion of this beamforming process may occur in the transducer electronics 110 and the rest of the process may occur in the system beamformer 118. For example, the processor 110/118 further includes a plurality of beam former processors (not shown), each beam former processor including a plurality of sub-array processors (not shown), each sub-array processor including at least one phase-adjuster (not shown) and a summer (not shown). Each phase-adjuster in the transducer electronics 110 is responsive to the signal generated in response to a received echo by each macro-element 218A-F to shift the signal by a respective phase angle and to apply the shifted signal to the summer. Each summer in the transducer electronics 110 is the output of a sub-array 210 beamformer processor. Each phase-adjuster in the system beamformer 118 is responsive to the signal generated in response to a received echo by each system channel 108 to shift the signal by a respective phase angle and to apply the shifted signal to the summer. Each phase adjuster is dynamically updatable during dynamic focusing of the processor 110/118. Each of the summers supplies a summed shifted signal from this beam former processor.

In one embodiment using sub-aperture mixing, the phase angles for any one of the sub-array processors form a sum substantially equal to zero. In another embodiment using sub-aperture mixing, each digital beamformer processor delays the respective sub-array signal by a respective time delay, and the phase angles for any one of the sub-array processors are independent of the time delay of the respective digital beamformer processor. In yet another embodiment using sub-aperture mixing, each digital beamformer processor delays the respective sub-array signal by a respective time delay, and wherein time resolution of the time delays is substantially as fine as time resolution of the phase angles. In yet another embodiment using sub-aperture mixing, the digital beamformer processors are characterized by a focusing update rate; and wherein the phase angles of the phase adjusting elements are updated at a slower rate than the focusing update rate.

In an embodiment using channel sharing/multiplexing techniques, such as time division multiplexing (“TDM”) or frequency division multiplexing (“FDM”), the processor 110/118 is further operative to combine one signal with another signal generated to cause at least one macro-element 218A-218F to form a beam or receive an echo and convey the combined signals over one of the plurality of cable conductors 116 (system channels 108), the individual signals being recoverable from the combination upon receipt. The processor 110/118 may combine the signals either using TDM or FDM. In one embodiment, each of the transducer electronics 110 and beamformer 118 include corresponding multiplexers/demultiplexers (not shown) which combine the signals for transmission and separate the signals upon receipt. In TDM, each signal occupies one or more time slots sub-divided from the overall bandwidth of the channel 108, as was described above. In FDM, each signal occupies a particular frequency sub-divided from the overall bandwidth.

For example, the system 100 may include a transducer 104 which includes (a) an array 202 of transducer elements 214 that transmit an ultrasonic beam at an object of which an image is to be formed, in a transmit mode, and receive the ultrasound reflected by the object in a receive mode; transducer electronics 110 which drive the macro-elements 218A-F with transmit pulses at individually specified starting times in the transmit mode, the transducer electronics 110 including an address decoder (not shown) and, for each macro-element 218A-F, a transmit pulser (not shown) connected to the address decoder. The transducer electronics 110 further include a multiplexer (not shown) which in the receive mode multiplexes groups of signals from the macro-elements 218A-F and feeds each group of transducer signals to a corresponding signal output. The system unit 102 includes a de-multiplexer (not shown) wherein the address decoder is connected to the system unit 102 via address lines (not shown) for transmitting addresses of the macro-elements 218A-F to be driven, and wherein the transmit pulsers are connected to the system unit 102 via common starting-time lines (not shown) for transmitting the starting-times for the transmit pulses, and wherein the signal outputs of the multiplexer and corresponding signal inputs of the de-multiplexer are connected via corresponding signal lines that carry the corresponding groups of transducer signals. Alternately the transmit means (not shown) can be located in the configurable array 114 where there is one set of transmit means circuits (not shown) for each transducer element 214.

In another example, the system 100, for transmitting, in a transmit mode, an ultrasonic beam at an object of which an image is to be formed, and for receiving, in a receive mode, the ultrasound reflected by the object, an imaging signal generating circuit (not shown) is provided. The imaging signal generating circuit includes an array 202 of transducer elements 214 capable of transmitting the ultrasonic beam and receiving the ultrasound reflected by the object and transducer electronics 110 electrically connected via separating filters (not shown), in the transmit mode, to each transducer element 214 in the array 202 of transducer elements. The transducer electronics 110 include a transmit pulser (not shown) for each macro-element 218A-F, provides phase-delayed driving of the transducer elements 214 during transmit mode, and having an address decoder (not shown) which is connected to each of the transmit pursers, the address decoder and the transmit pulsers being electrically interconnected via a plurality of selector lines (not shown). The imaging signal generating circuit further includes a multiplexer (not shown) electrically connected via separating filters (not shown), in the receive mode, to each macro-element 218A-F in the array 202 of transducer elements, wherein the multiplexer receives transducer signals representing the reflected ultrasound from the object. The system unit 102 includes a de-multiplexer, wherein the system unit 102 provides signal processing of the transducer signals received from the macro-elements 218A-F during the receive mode. The system 100 also includes a plurality of starting-time lines (not shown) electrically connecting the system unit 102 to the transmit pulsers, wherein the starting-time lines transmit starting times for transmit pulses and a plurality of signal lines electrically connecting the multiplexer and the de-multiplexer, wherein the signal lines carry a group of transmitted multiplexed signals from the transducers; and at least one address line electrically connecting the address decoder to the base unit.

Using TDM or FDM techniques with a configurable array 114, the processor 110/118 may combine signals to form and actuate multiple macro-elements 218A-218F, and convey the combined signals over one of the plurality of system channels so as to recover the signals at the receiving end.

For example, the disclosed embodiments may be used to perform dynamic element combining. For an array 202 of a fixed number of transducer elements 214, this scheme groups element signals to reduce the required communication bandwidth when using TDM signaling. The elements 214 are grouped in pairs as macro-elements 218A-218F by summing neighbors perpendicular to the beam angle. As the beam angle changes, different pairs are summed, i.e. different macro-element 218A-218F groups 220 are formed, effectively changing the apparent shape of the elements 214 for the system-based beam-former 118.

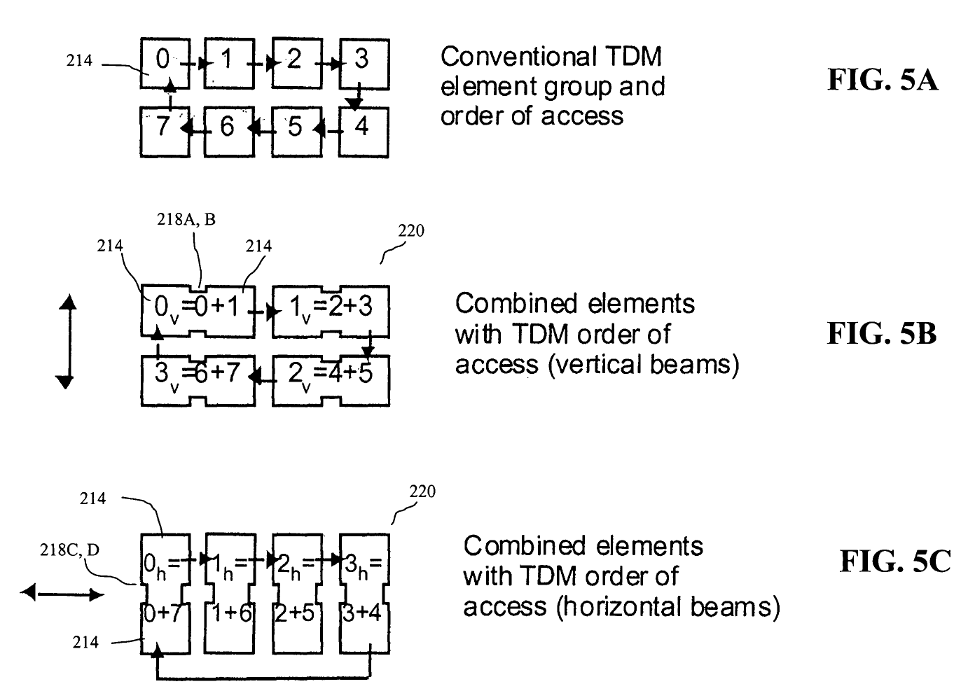

FIGS. 5A-5C show conventional TDM where 8 elements 214 are time multiplexed. In FIG. 5A, control signals for the eight elements 214 shown are sent to the transducer 104 separately. The sample rate for each element is ⅛^{th} of the multiplexing clock rate. If the clock rate is 80 MHz, the sample rate for each element 214 is 10 MHz and the space between analog samples is 12.5 nanoseconds.

FIGS. 5B and 5C show two versions of combining elements 214. Should a beam be steered more vertically than horizontally, the group 220 of FIG. 5B is used. Should a beam be steered more horizontally than vertically, the group 220 of FIG. 5C should be used.

Using TDM, the sample rate for each combined element is ¼^{th} the multiplexing clock rate. If the clock rate is 80 MHz, the sample rate for each combined element is 20 MHz and the space between analog samples is 12.5 nanoseconds. If the clock rate is 40 MHz, the sample rate for each combined element 218A-218F is 10 MHz and the space between analog samples is 25 nanoseconds. Hence, it is possible to increase the sample rate or increase the time between samples, or any combination of these positive benefits.

Using FDM, the bandwidth can be effectively doubled using the same channel 108 spacing, the channel 108 spacing can be increased, or the bandwidth and channel 108 spacing can remain the same and the multiplexer uses less overall bandwidth.

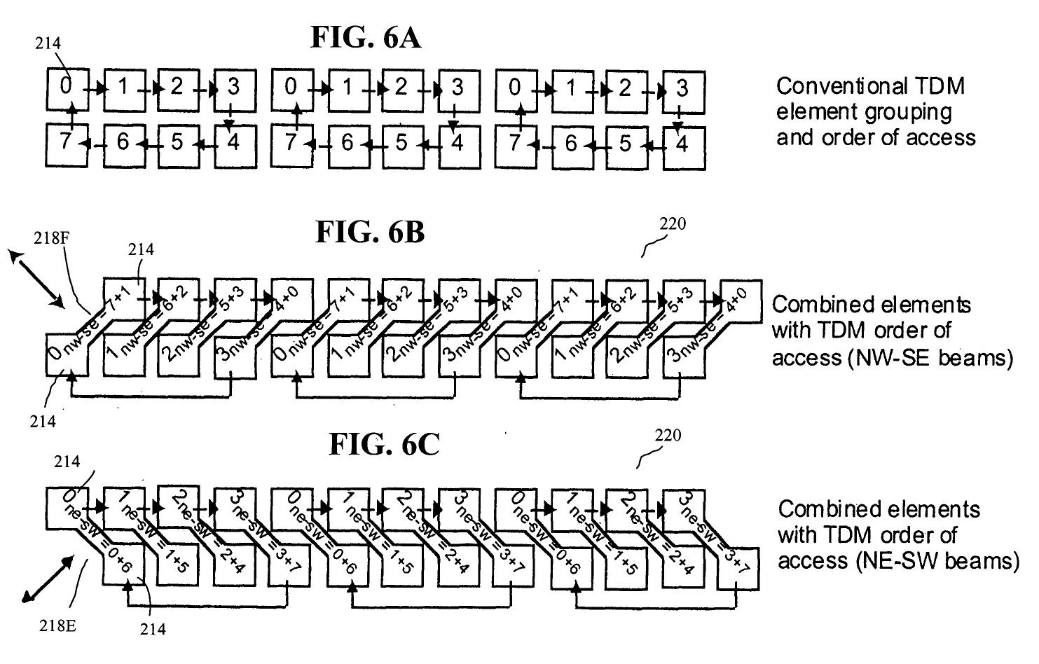

In another embodiment, shown in FIGS. 6A-6C, dynamic element combining is implemented across element groups. In this scheme, the dynamic element combining described above is combined with one aspect of sub-array 210A-210D remapping across element groups to allow element combining to better support beam steering between the 0, 90, 180 and 270 degree locations. This is accomplished by providing one additional expander output to a neighbor and one additional expander input from a neighbor. FIG. 6A depicts conventional TDM element grouping an order of access. FIGS. 6B and 6C show two versions of combining elements depending on the beam angle, i.e. Northwest/Southeast (FIG. 6B) or Northeast/Southwest (FIG. 6C). FDM may be used instead of TDM.

Element combining, within or across element multiplexing groups using TDM or FDM multiplexing, maximizes multiplexer performance by increasing time or frequency between samples and/or by increasing the element signal sample rate or bandwidth.

It is therefore intended that the foregoing detailed description be regarded as illustrative rather than limiting, and that it be understood that it is the following claims, including all equivalents, that are intended to define the spirit and scope of this invention.

1. A multi-dimensional transducer array system for ultrasonically scanning a three dimensional volume, the system comprising:
a multi-dimensional array of configurable sub-sets, each configurable sub-set comprising a plurality of transducer elements, each of the transducer elements capable of being selectively interconnected with at least another of the transducer elements to form at least one macro-element of a plurality of macro-elements;
a plurality of system channels coupled with the transducer elements; and
a processor coupled with the multi-dimensional array and the plurality of system channels and operative to configure the interconnection of the plurality of transducer elements of at least two of the plurality of configurable sub-sets to form the at least one macro-element for each of the at least two of the plurality of configurable sub-sets as a function of a beam position, the at least one macro-element of a first of the at least two of the plurality of configurable sub-sets operative to generate a first signal and the at least one macro-element of a second of the at least two of the plurality of configurable sub-sets operative to generate a second signal, and wherein the processor is further operative to combine the first and second signals for communication over one of the plurality of system channels.

2. The system of claim 1, wherein the processor is further operative to configure the interconnection as a function of beam position.

3. The system of claim 1, wherein the processor is further operative to configure the interconnection according to a first configuration for transmit operations and according to a second configuration for receive operations.

4. The system of claim 1, wherein the processor us further operative to configure the interconnection to form a sparse array.

5. The system of claim 1, wherein the processor combines the first and second signals using a beam forming process selected from the processes of walking aperture multiplexing, partial beam forming, sub-aperture mixing, time division multiplexing and frequency division multiplexing.

6. The system of claim 1, wherein the processor is further operative to select the second signal generated by the second of the at least two of the plurality of sub-sets sequentially after selecting the first signal generated by the first of the at least two of the plurality of sub-sets.

7. The system of claim 1, wherein the processor is operative to combine the first signal with the second signal by delaying the first signal with respect to the second signal and summing the delayed first signal with the second signal.

8. The system of claim 1, wherein the processor is operative to combine the first signal with the second signal by altering the phase of the first signal with respect to the second signal and summing the altered first signal with the second signal.

9. The system of claim 1, wherein the processor is further operative to combine the first signal with the second signal so as to be able to recover each of the first and second signals from the combined first and second signals.

10. The system of claim 9, wherein the processor is operative to combine the first and second signals using time division multiplexing.

11. The system of claim 9, wherein the processor is operative to combine the first and second signals using frequency division multiplexing.

12. A multi-dimensional transducer array system for ultrasonically scanning a three dimensional volume, the system comprising:
a multi-dimensional array of transducer elements;
a switching network coupled with the multi-dimensional array and operative to selectively interconnect the transducer elements into a plurality of macro-element groups, the switching network further including a plurality of switch sets, each of the plurality of switch sets associated with a sub-set of transducer elements of the array of transducer elements, each of the plurality of switch sets including a plurality of switches operable to selectively interconnect at least one transducer elements of the associated sub-set into a macro-element, the plurality of macro-element groups comprising at least one of the macro-elements formed by at least one of the plurality of switch sets;
a plurality of system channels operable to be connected with respective macro-elements, each of the plurality of system channels being associated with at least one of the plurality of switch sets;
wherein at least two of the plurality of switches of at least one of the plurality of switch sets for forming the macro-element coupled with each system channel; and
a processor coupled with the plurality of system channels and the switching network and operative to cause the switching network to form a first macro-element group of the plurality of macro-element groups and generate a first signal to cause at least one macro-element of the first macro-element group to one of form a first beam and receive a first echo.

13. The system of claim 12, wherein each of the plurality of switches comprises a micro-mechanical based switch.

14. The system of claim 13, wherein each of the transducer elements comprises a micro-mechanical based transducer element.

15. The system of claim 14, further comprising a substrate, the substrate including both the plurality of switches and the transducer elements.

16. The system of claim 12, wherein the sub-set of transducer elements associated with one of the plurality of switch sets may overlap with the subset of transducer elements of another of the plurality of switch sets.

17. The system of claim 12, wherein the plurality of switches is operable to selectively interconnect at least two transducer elements of the associated sub-set in the macro-element.

18. The system of claim 12, wherein at least two elements of the sub-set of transducer elements are adjacent to one another.

19. The system of claim 18, wherein the at least two elements of the sub-set are diagonally adjacent to one another.

20. The system of claim 12, wherein the at least two transducer elements are selectively interconnected based on a desired steering angle of the first beam.

21. The system of claim 12, wherein the processor is further operative to cause the switching network to form the first macro-element group when generating the first signal to cause the at least one macro-element to form the first beam and to cause the switching network to form a second macro-element group, different from the first macro-element group, when generating a second signal to cause at least one macro-element of the second macro-element group to receive the first echo.

22. The system of claim 12, wherein the processor is further operative to cause the switching network to form the first macro-element group when generating the first signal to cause the at least one macro-element to form the first beam and to cause the switching network to form a second macro-element group, different from the first macro-element group, when generating a second signal to cause at least one macro-element of the second macro-element group to form a second beam.

23. The system of claim 12, wherein each of the plurality of macro-element groups is characterized by an apparent acoustic origin, the processor being further operative to compensate for the apparent acoustic origin of the first macro-element group.

24. The system of claim 12, wherein the first macro-element group comprises a sparse array pattern of the macro-elements.

25. The system of claim 12, wherein the processor generates the signal according to a beam forming process selected from the processes of walking aperture multiplexing, partial beam forming, sub-aperture mixing, time division multiplexing, and frequency division multiplexing.

26. The system of claim 12, wherein the array of transducer elements is further divided into a plurality of sub-arrays of transducer elements, each of the plurality of sub-arrays comprising at least one of the sub-sets, the first macro-element group comprising macro-elements of a first sub-array, the processor being further operative to form a second macro-element group comprising macro-elements of a second sub-array and generate a second signal to cause at least one macro-element of the second macro-element group to one of form a second beam and receive a second echo, after generating the first signal.

27. The system of claim 12, wherein the multi-dimensional array comprises N×M transducer elements, there being M columns of N transducer elements, wherein M and N are integers;
the processor including a transmitter for generating the first signal to cause the at least one macro-element to form the first beam and a receiver for generating the first signal to cause the at least one macro-element to receive the first echo;
the processor being further operative to couple the transmitter with a plurality of sub-arrays of N×X transducer elements, where X is an integer less than M, each of the plurality of sub-arrays comprising at least one of the sub-sets, so as to cause each of the at least one sub-set of each sub-array to form one of the plurality of macro-element groups and cause at least one of the macro-elements of the one of the plurality of macro-element groups to form a beam;
the processor being further operative to sequentially couple the transmitter and receiver to each of the sub-arrays so as to enable reception by the receiver of echoes from an elongated sector volume.

28. The system of claim 12, wherein the processor is further operative to combine the first signal of a first of the at least one macro-element of the first macro-element group with a second signal of a second of the at least one macro-element of the first macro-element group and convey the combined first and second signals over one of the plurality of system channels.

29. The system of claim 28, wherein the processor is further operative to combine the first signal with the second signal by delaying one of the first and second signals with respect to the other of the first and second signals and summing the delayed one of the first and second signals with the other of the first and second signals.

30. The system of claim 28, wherein the processor is further operative to combine the first signal with the second signal by adjusting the phase of one of the first and second signals with respect to the other of the first and second signals and summing the phase adjusted one of the first and second signals with the other of the first and second signals.

31. The system of claim 12, wherein:
the array of transducer elements is further divided into a plurality of sub-arrays of transducer elements, each of the plurality of sub-arrays comprising at least one of the sub-sets;
the processor further comprising a plurality of intra-group transmit processors coupled with the plurality of sub-arrays, operative to cause the generation of the first signal to form the first beam directed into a region of interest;
the array of transducer elements further comprising a receive array of transducer elements, the receive array including at least one of the sub-sets;
the processor further comprising a receive beamformer, the receive beamformer including the plurality of system channels, each of the plurality of system channels including a beamformer delay operative to synthesize receive beams from the received first echo by delaying the first signal received from the at least one macro-element of the receive array;
the receive beamformer further including a beamformer summer operative to receive and sum the first signal from the plurality of system channels and an image generator operative to form an image of the region of interest based on the first signal received from the receive beamformer.

32. The system of claim 12, wherein:
the processor further comprises a plurality of beam former processors, each beam former processor comprising a plurality of sub-array processors, each sub-array processor comprising at least one phase-adjuster and a summer, each phase-adjuster responsive to the first signal of the first received echo of each of the at least one macro-element to shift the first signal by a respective phase angle and to apply the shifted first signal to the summer, each phase adjuster dynamically updatable during dynamic focusing of the processor, each of the summers supplying a summed shifted first signal to the associated beam former processor.

33. The system of claim 12, wherein the processor is further operative to combine the first signal with a second signal generated to cause at least one macro-element of the first macro-element group to one of form a second beam and receive a second echo, the processor further operative to convey the combined first and second signals over one of the plurality of system channels, the first and second signals being recoverable from the combined first and second signal.

34. The system of claim 33, wherein the processor is operative to combine the first and second signals using time division multiplexing.

35. The system of claim 33, wherein the processor is operative to combine the first and second signals using frequency division multiplexing.

36. The system of claim 33, wherein the combined first and second signals is transmitted over a period of time, at least a portion of the first signal occupying a first portion of the period of time and at least a portion of the second signal occupying a second portion of the period of time.

37. The system of claim 33, wherein the combined first and second signals comprise the first signal having a first frequency and the second signal having a second frequency different from the first frequency.

38. The system of claim 12, wherein the processor is further operative to combine the first signal with a second signal generated to cause at least one macro-element of a second macro-element group to one of form a second beam and receive a second echo, the processor being further operative to convey the combined first and second signals over one of the plurality of system channels, the first and second signals being recoverable from the combined first and second signal.

39. The system of claim 38, wherein the processor is operative to combine the first and second signals using time division multiplexing.

40. The system of claim 38, wherein the processor is operative to combine the first and second signals using frequency division multiplexing.

41. The system of claim 38, wherein the combined first and second signals is transmitted over a period of time, at least a portion of the first signal occupying a first portion of the period of time and at least a portion of the second signal occupying a second portion of the period of time.

42. The system of claim 38, wherein the combined first and second signals comprise the first signal having a first frequency and the second signal having a second frequency different from the first frequency.

43. In a multi-dimensional transducer array system, a method for ultrasonically scanning a three dimensional volume, the method comprising:
providing a multi-dimensional array of configurable sub-sets, each configurable sub-set comprising a plurality of transducer elements, each of the transducer elements capable of being selectively interconnected with at least another of the transducer elements to form at least one macro-element of a plurality macro-elements;
providing a plurality of system channels coupled with the transducer elements;
configuring the interconnection of the plurality of transducer elements of at least two of the plurality of sub-sets to form the at least one macro-element for each of the at least two of the plurality of sub-sets as a function of a beam position, the at least one macro-element of a first of the at least two of the plurality of sub-sets generating a first signal and the at least one macro-element of a second of the at least two of the plurality of sub-sets generating a second signal; and
combining the first and second signals and communicating the combined first and second signals over one of the plurality of system channels.

44. The method of claim 43, the configuring further comprising configuring the interconnection as a function of beam position.

45. The method of claim 43, the configuring further comprising configuring the interconnection according to a first configuration for transmit operations and according to a second configuration for receive operations.

46. The method of claim 43, the configuring further comprising configuring the interconnection to form a sparse array.

47. The method of claim 46, wherein the configuring further comprises configuring the interconnection to form a second sparse array subsequent to configuring the interconnection to form a first sparse array, the first sparse array being different from the second sparse array.

48. The method of claim 43, wherein the combining further comprising combining the first and second signals using a beam forming process selected form the processes of walking aperture multiplexing, partial beam forming, sub-aperture mixing, time division multiplexing and frequency division multiplexing.

49. The method of claim 43, further comprising causing the second of the at least two of the plurality of sub-sets to generate the second signal sequentially after causing the first of the at least two of the plurality of sub-sets to generate the first signal.

50. The method of claim 43, the combining further comprising combining the first signal with the second signal by delaying the first signal with respect to the second signal and summing the delayed first signal with the second signal.

51. The method of claim 43, the combining further comprising combining the first signal with the second signal by altering the phase of the first signal with respect to the second signal and summing the altered first signal with the second signal.

52. The method of claim 43, the combining further comprising combining the first signal with the second signal so as to be able to recover each of the first and second signals from the combined first and second signals.

53. The method of claim 52, the combining further comprising combining the first and second signals using time division multiplexing.

54. The method of claim 52, the combining further comprising combining the first and second signals using frequency division multiplexing.

55. In a multi-dimensional transducer array system, a method for ultrasonically scanning a three dimensional volume, the method comprising:
providing a multi-dimensional array of transducer elements;
providing a switching network coupled with the multi-dimensional array;
selectively interconnecting the transducer elements, using the switching network, into a plurality of macro-element groups, the switching network further including a plurality of switch sets, each of the plurality of switch sets associated with a sub-set of transducer elements of the array of transducer elements, each of the plurality of switch sets including a plurality of switches;
selectively interconnecting, using the plurality of switches, at least one transducer elements of the associated sub-set into a macro-element, the plurality of macro-element groups comprising at least one of the macro-elements formed by at least one of the plurality of switch sets;
providing a plurality of system channels operable to be connected with respective macro-elements, each of the plurality of system channels being associated with at least one of the plurality of switch sets;
connecting at least two of the plurality of switches of at least one of the plurality of switch sets for forming the macro-element with each system channel; and
forming a first macro-element group of the plurality of macro-element groups and generating a first signal to cause at least one macro-element of the first macro-element group to one of form a first beam and receive a first echo.

56. The method of claim 55, wherein said selective interconnecting using the plurality of switches further comprises selectively interconnecting at least two transducer elements of the associated sub-set into the macro-element.

57. A multi-dimensional transducer array system for ultrasonically scanning a three dimensional volume, the system comprising:
a multi-dimensional array of configurable sub-sets, each configurable sub-set comprising a plurality of transducer elements, each of the transducer elements capable of being interconnected with at least another of the configurable transducer elements to form at least one macro-element of a plurality macro-elements;
a plurality of system channels coupled with the configurable transducer elements; and
means for configuring the interconnection of the plurality of transducer elements of at least two of the plurality of sub-sets to form the at least one macro-element for each of the at least two of the plurality of sub-sets as a function of a beam position, the at least one macro-element of a first of the at least two of the plurality of sub-sets operative to generate a signal and the at least one macro-element of a second of the at least two of the plurality of sub-sets operative to generate a second signal, and means for combining the first and second signals for communication over one of the plurality of system channels.

58. A multi-dimensional transducer array system for ultrasonically scanning a three dimensional volume, the system comprising:
a multi-dimensional array of transducer elements;
means for selectively interconnecting the transducer elements into a plurality of macro-element groups including a plurality of switch sets, each of the plurality of switch sets associated with a sub-set of transducer elements of the array of transducer elements, each of the plurality of switch sets including a plurality of switch means for selectively interconnecting at least two transducer elements of the associated sub-set into a macro-element, the plurality of macro-element groups comprising at least one of the macro-elements formed by at least one of the plurality of switch sets;
a plurality of system channels operable to be connected with respective macro-elements, each of the plurality of system channels being associated with at least one of the plurality of switch sets;
wherein at least two of the plurality of switch means of at least one of the plurality of switch sets for forming the macro-element connect with each system channel; and
means for causing the switching network to form a first macro-element group of the plurality of macro-element groups and generate a first signal to cause at least one macro-element of the first macro-element group to one of form a first beam and receive a first echo.
